(19) United States
(12) Patent Application Publication
(10) Pub. No.: US 2014/0249517 A1
(43) Pub. Date: Sep. 4, 2014

(54) METHOD AND APPARATUS FOR ELIMINATING ATHEROSCLEROSIS FROM A REGION OF THE ARTERIAL TREE

(71) Applicant: Ajoy I. SINGH, Maharashtra (IN)

(72) Inventor: Ajoy I. Singh, Maharashtra (IN)

(21) Appl. No.: 14/352,235

(22) PCT Filed: Oct. 12, 2012
(86) PCT No.: PCT/IN2012/000682

§ 371 Date: Apr. 16, 2014

(30) Foreign Application Priority Data
Oct. 17, 2011 (IN) 2896/MUM/2011

(51) Int. Cl. A61B 18/24

(52) U.S. Cl. CPC A61B 18/245; A61B 2018/00404
USPC 606/7

(57) ABSTRACT

The present invetion provides a method and apparatus for elimination of atherosclerosis from an artery. According to the present invetion the diseased artery is approached from external side and ablate is approached in such a way that incision/cut pass through tunica adventitia and tunica media of the diseased artery and a fibrous capsule of the atherselerosis. On ablation/incision, the contents plaques are exposed to the natural defense of the body and are destroyed by the natural defense system. The plaque escaping out of the artery on the external surface of the artery may be wiped or washed away with saline during or after the ablation procedure. Then, natural healing of artery is allowed which eliminates atherosclerosis thoroughly.

### FIELD OF THE INVENTION

The present invention relates to a method and apparatus for eliminating atherosclerosis from an artery.

### PRIOR ART OF THE INVENTION

It is known that a normal artery (70) as shown in FIG. 1A has a lumen (60) defined by a three layered structure namely innermost layer called as tunica intima (10), middle layer called as tunica media (20) and outermost layer called as tunica adventitia (30). Atherosclerosis is a disorder of arteries that afflicts a large proportion of humanity. The disease is characterized by the blockages in arteries develop due to accumulation of cholesterol, fats and other chemicals between the tunica intima (10) and the tunica media (20) of the artery wall as shown in FIG. 1B. This accumulation is enveloped in a thin fibrous capsule (40). The fibrous capsule (12) with the accumulations of cholesterol, fats and other chemicals are often collectively referred to as “atherosclerotic or atheromatous plaque” (50). This plaque (50) also contains cells of various types, predominantly macrophages, giant cells and smooth muscle cells. These cells are a consequence of the inflammatory nature of the disease process afflicting the wall of the artery. Thus, atherosclerosis is characterized by enlargement of the artery wall as shown in FIG. 1B. Principally restriction is due to atherosclerotic plaque bulging into the inner space (lumen) of the artery. This leads to increased arterial wall stiffness and reduced internal diameter of the artery, and consequently reduced blood flow though the artery. If these contents are released to the inside of the vessel wall it may lead to a heart attack or a brain stroke due to thrombosis (clotting of blood inside the artery). The degree of atherosclerosis is measured as an increase in Intima Media Thickness (“IMT”).

Further, it has since been recognized that atherosclerosis is a systemic disease and blockages (stenoses) a local manifestations of the disease process which may also widespread and distributed over a region of the arterial tree as shown in FIG. 2. The disease manifests itself in an angiogram as a local narrowing/blockage, but pathology studies confirm the existence of disease in areas that are not narrowed as well.

In real life, the biological process accompanying atherosclerosis is a lot more complex, including a self-healing mechanism of the human or animal body that attempts to minimize the constriction of the artery, called stenosis in medical terminology. The self-healing mechanism functions by “remodeling” the artery. The constituents of these prolonged depositions, called atheroma, include macrophage cells, cellular debris of dead cells and living cells, as well as the fibrous tissue covering of the atheroma itself. Over time, calcification can also occur between the atheroma layer and the underlying smooth muscle cell layer of the vessel wall.

Numerous medical equipments and techniques are available today for unblocking coronary arteries blocked by the atheromatous plaque. Chief among them are balloon angioplasty, Stents, rotational atherectomy, directional atherectomy and transluminal extraction atherectomy.

During an actual medical procedure, each of above techniques typically uses catheter. For guidance, a guide wire is typically inserted first before the catheter. The catheter is then passed over the guide wire to reach the target area. The approach is through the artery lumen. All these procedure may cause some degree of injury to the inner-most lining, the tunica intima. Hence, many of these procedures have a high rate of re-blockage due to cellular proliferation, which follows any injury to the tunica intima. The advent of drug eluting stents has reduced this incidence significantly, but limitations remain.

The U.S. Pat. No. 6,669,686 granted to the present inventor discloses a method that avoids the through the artery approach, more particularly a method for reducing the thickness of an arterial wall by ablation of the exterior of the artery wall by laser ablating or removing the exterior layer of the arterial wall the tunica intima and inner layers of the tunica media are protected from damage. The flexibility of the artery is improved due to the reduced effective wall thickness after ablation thus relieving stenosis and improving blood flow through the artery. However, this procedure treats only the local condition, the stenosis and not the full extent and distribution of the disease. Moreover, this procedure increases flexibility of artery and does not eliminate the atherosclerosis.

Treatment of atherosclerosis by all known present technologies is carried out or suggested to be carried out only when blockage of the vessel is more than 50% of internal diameter of the artery as the said technologies are nearly ineffective in treating early and about mid-stage plaque formation. This is particularly troublesome in view of the fact that mid-stage vulnerable plaque formation with minimum lumen intrusion is now clinically considered to be even more dangerous owing to its tendency to rupture spontaneously, leading to immediate and severe heart attack or even instant death.

Atherosclerosis spreads over artery including regions of the arterial tree. The disease process leads to wall thickening, but poses no real obstruction to the blood flow in the artery. In some small localized regions the disease tends to progress more rapidly leading to an obstruction to the flow of blood. Currently all treatments target the obstruction (obstructive disease) solely and fresh obstructions keep arising from the underlying disease, requiring repeated treatments. Artery having atherosclerosis are referred as ‘diseased artery’ hereinafter.

There is therefore a need to eliminate the said disease, so that new obstructions do not arise at a later date.

### SUMMARY OF THE INVENTION

Accordingly, in one aspect the present invention provides a method for elimination of atherosclerosis from an artery. According to the method of the present invention, said elimination of atherosclerosis is achieved by exposing atheromatous plaque of the atherosclerosis to the natural defense system of body, comprising of scavenger white blood cells like macrophages, monocytes etc., thereby, eliminating atherosclerosis.

According to preferable embodiment of the present invention, the method for elimination of atherosclerosis from an artery includes step of approaching a diseased artery from external side, ablating artery wall including ablation of tunica adventitia and tunica media of the artery and a fibrous capsule of the atherosclerosis and exposing atheromatous plaque of the atherosclerosis to the natural defense system of body thereby eliminating atherosclerosis.

Advantageously, the method comprises a step of surveying thickness of the arterial wall and length of the atherosclerosis prior to ablating and during ablating to calculate depth of ablating incision required to expose atheromatous plaque.

According to the present invention, the step ablating includes providing a single or multiple longitudinal incisions. Advantageously, longitudinal incision can be a continuous or discontinuous incision extending from one end to another end of atherosclerosis.

According to the preferable embodiment of the present invention ablation is carried out by laser.

The present invention also provides an apparatus for eliminating atherosclerosis from an artery, comprising a surveying means for surveying a thickness of an arterial wall and length of the atherosclerosis spread in the artery for defining ablating target on the surface of the artery and calculating depth of ablating incision required from external surface of the artery to a fibrous capsule of the atherosclerosis for exposing atheromatous plaque to a natural defense system, an ablating apparatus and a controller means for controlling the ablation of the arterial wall by the ablating apparatus. According to the present invention, The ablating apparatus comprises an ablating means for ablating the arterial wall externally, a robotic arm for adapting the ablating means for carrying out ablation of arterial wall. The controller means interacts between said surveying means and the ablating apparatus and is being responsive to a signal from said surveying means, the signal relating to the depth and length of ablating incision required to expose atheromatous plaque to the natural defense system of the body helping to eliminate atherosclerosis from the artery.

According to the preferable embodiment of the present invention, the surveying means comprises two transverse surveying scanners adapted on the robotic arm before and after ablating means for monitoring ablating process in real time.

According to the preferable embodiment of the present invention, the ablating means a laser. Alternatively, the ablating means can be a mechanical ablation device including at least one blade mounted on at least one pressure sensing transducer.

According to the present invention, the laser is a femto-second pulsed laser having pulses preferably between 10 to 750 fs duration.

### DEFINITIONS

Diseased artery: An artery having atherosclerosis and also includes artery tree having atherosclerosis spread over it.

Natural defense of body: It is considered in its general term that immune system of the body recognizes and destroys foreign substances and organisms that enter the body with the help of body defense cells—the WBC's including macrophages, monocytes, neutrophils etc.

Natural healing of body: It is a process of the restoration of health to an unbalanced, diseased or damaged organism. It is the process by which the cells in the body regenerate and repair to reduce the size of a damaged area and replace it with new living tissue for example Healing of skin after a cut; or Healing of a fractured bone.

### BRIEF DESCRIPTION OF THE ACCOMPANYING DRAWINGS

To illustrate the invention, a preferred embodiment thereof will now be described with reference to the accompanying drawings (which in no way restrict the scope of the invention and are for the purpose of illustration only) in which:

FIG. 1 shows a partially sectioned schematic view of an artery wherein
FIG. 1A shows a normal artery; and
FIG. 1B shows a diseased artery that is artery having thickened wall due to atherosclerosis;

FIG. 2 shows a partially sectioned schematic view of a widespread and distributed atherosclerosis over a diseased arterial tree;

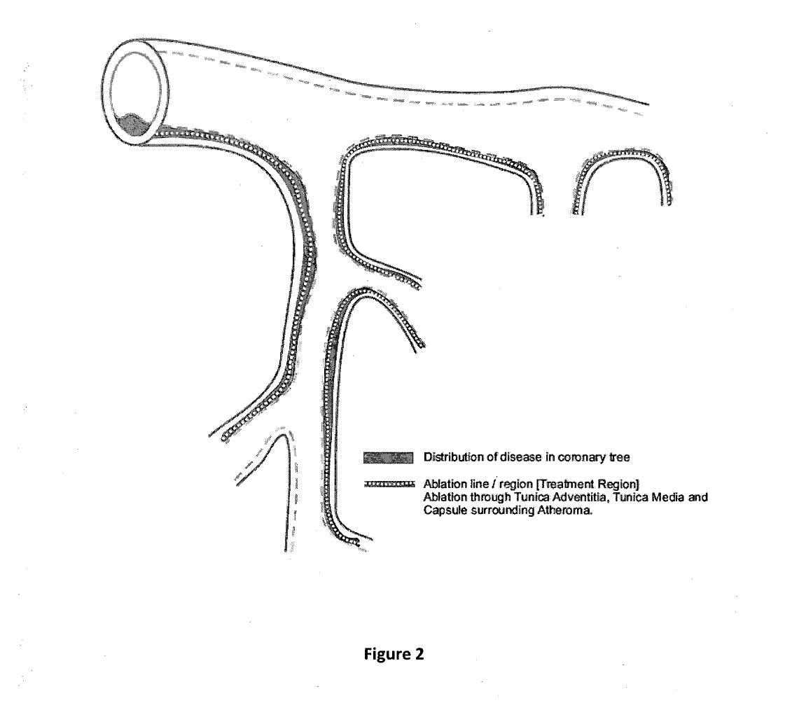

FIG. 3 shows a block diagram of an apparatus for elimination of atherosclerosis from the diseased artery according to the present invention;

FIG. 4 shows a schematic view of an ablating apparatus according to the present invention

FIG. 5 shows a longitudinal ablated incision of the diseased artery according to the present invention; and

FIG. 6 through 6A-6F shows a series of six cross sectioned schematic view of the artery wherein:
FIG. 6A is cross sectional view of diseased artery,
FIG. 6B-6E are cross sectional view of diseased artery being treated according to the present invention, and
FIG. 6F is cross-sectional view of a treated and healed artery.

### DESCRIPTION OF THE INVENTION

In preferred general term, the present invention provides a method for eliminating atherosclerosis from a diseased artery and an apparatus thereof wherein diseased artery is approached externally and cut upto a fibrous capsule of the atherosclerosis including the fibrous capsule. On ablation/incision, the contents plaques are exposed to the natural defense of the body and are destroyed by the natural defense system. The plaque escaping out of the artery on the external surface of the artery may be wiped or washed away with saline during or after the ablation procedure. Then, natural healing of artery is allowed which eliminates atherosclerosis thoroughly. The ablation is carried out in such a way that the innermost layer of the artery namely tunica intima remains undisturbed.

According to the preferred embodiment of method of the present invention, the first step comprises surveying the diseased artery with atherosclerosis with the surveying means. According to the present invention, the step of surveying of the diseased artery includes measuring thickness of the arterial wall and length of the atherosclerosis prior to ablating for calculating depth and length of ablating incision required to expose atheromatous plaques. According to the further embodiment of the present invention, the surveying step also includes identifying a thickest portion of arterial wall of the diseased artery as a target for carrying out ablation according to the present invention.

The second step comprises ablating the tunica adventitia and tunica media of the artery and a fibrous capsule in contact with, the tunica media so that atheromatous plaques of the atherosclerosis in the fibrous capsule get exposed to the natural defense system of the body thereby eliminating the atherosclerosis from the artery. Preferably, thickest portion of the arterial wall is ablated.

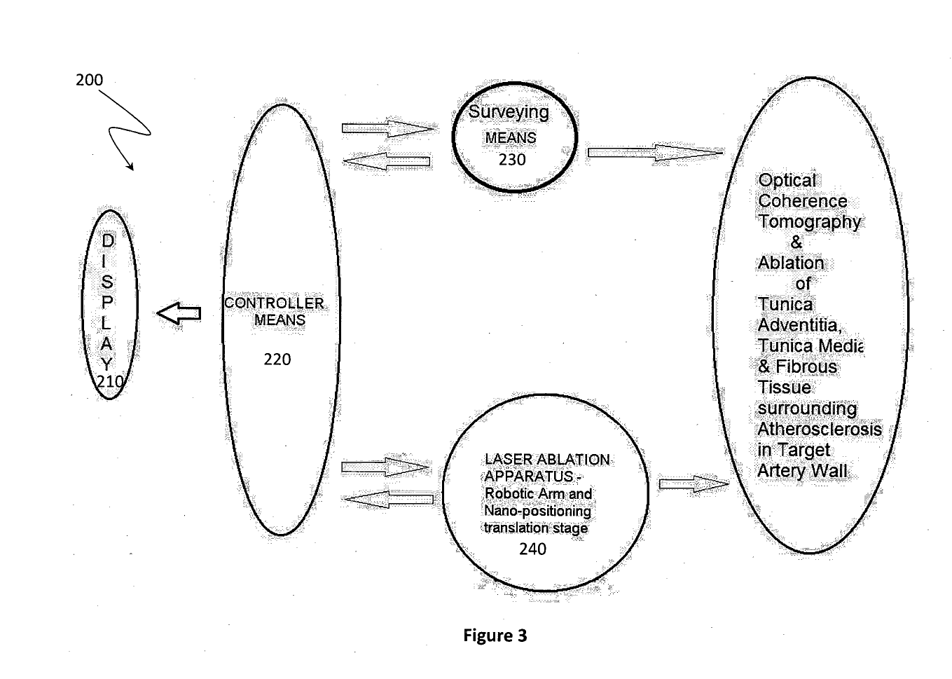

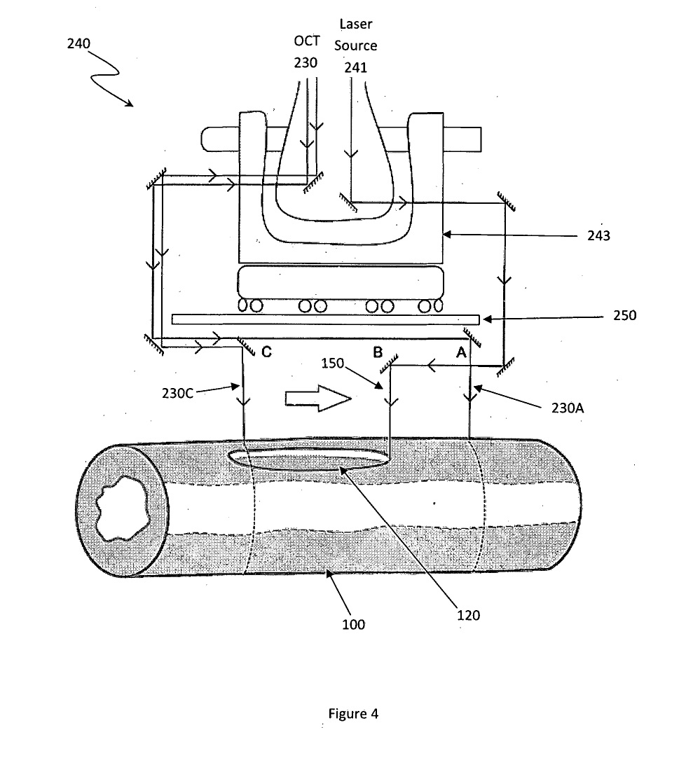

FIG. 3 is a block diagram illustrating an apparatus for elimination of atherosclerosis and FIG. 4 shows an ablating apparatus according to the present invention.

The apparatus (200) comprises a surveying means (230) for surveying the diseased artery (100), a controller means (220), a display (210) and an ablating apparatus (240).

The surveying means (230) is connected to the controller means (220). Suitable known techniques include ultrasound imaging, magnetic resonance imaging, electromagnetic radiation based tomographic imaging and photonic imaging can be used as the surveying apparatus (230) for surveying the artery wall thickness. The list is by no means exhaustive. Those skilled in the art will readily appreciate other techniques can be used. The surveying step should allow a precise mapping of the artery. Desirably the mapping will provide a three-dimensional (3D) image of the artery. Desirably, the surveying step is also performed in real time to allow monitoring and feedback for the subsequent steps.

In accordance with the preferred embodiments, the surveying means (230) is an imaging system using coherence optical tomography such as an Optical Coherence Tomography (OCT) system which includes an OCT beam scanner, which scans the diseased artery and provides the scanned data to the controller. The OCT is used for imaging the arterial wall of diseased artery in 3D prior to ablation. The 3D model of the artery which is analysed by the controller to determine the necessary additional ablations required to expose the atheromatous plaques of atherosclerosis to the natural defense system of body for complete elimination. The process of ablation/incision is also controlled on-line through the OCT. This allows very precise control of ablation (micro-machining) performed on the artery wall using a femto-second pulsed laser micro-machining system. The OCT uses a femto-second pulsed laser beam from the same laser source to produce high resolution images (+/−10 microns) of greater depth. The OCT system according to the present invention comprises one longitudinal beam scanner and two transverse beam scanners. The OCT scanners creates a 2D digital images which are stacked to produce a 3D image model of the diseased artery. As is well known in the art, the surveying scanner comprises a scanning beam which is reflected by reflective mirrors or travels through a optical path and scans the underlying tissue, including the artery. The reflected beam is analyzed to produce a three-dimensional (3D) digital model rendering of the artery. In accordance with the invention, the image data thereby produced is then used for calculating thickness of artery wall for accurate positioning and control of the ablating apparatus. The 3D digital image is also used for calculating the number of laser pulses required to be delivered at each 10 micron diameter target on the surface of the diseased artery. This ensures a uniform residual arterial wall thickness that pulsates in response to the blood pressure remains behind after the ablation. Preferably, the imaging system uses a low intensity infra red laser to produce the image of the underlying tissue. The produces a distance map and 3D model of the artery. The necessary positioning, movement and control of the ablating apparatus along with length and depth of ablation are computed using the image data for elimination of the atherosclerosis. The depth of ablation includes thickness of the tunica adventitia and tunica media of the artery and a fibrous capsule of the atherosclerosis in contact with the tunica media of the artery.

The display (210) for displaying the results of survey (image) is connected to the surveying means (110) for viewing by the surgeon. According to an embodiment, the display is a touch sensitive liquid crystal display screen displays the digital image captured by a video camera (not shown). The digital image in the display is used for guiding the laser ablating apparatus to specific segments/portions of the target diseased artery.

The ablating apparatus (240) is provided and desirably connected to the controller (220). The controller (220) precisely control the ablating apparatus to ensure the high degree of precision necessary for successful ablation. The controller (220) in combination with the surveying means (230) and the ablating apparatus (240) establishes a feed back loop. Thus, by feeding back data from the surveying means (230) to the controller (220) as it moves the ablating apparatus (240) thereby automated reciprocal ablation can be achieved.

The ablating apparatus (240) under control of the controller means (220) makes incisions of a depth in the arterial wall equal to thickness of tunica adventitia, tunica intima and fibrous capsule in contact with tunica intima so as to expose atheromatous plaques of the atherosclerosis to the natural defense system of body. On ablation/incision, the contents plaques are exposed to the natural defense of the body and are destroyed by the natural defense system. The plaque escaping out of the artery on the external surface of the artery may be wiped or washed away with saline during or after the ablation procedure. As the plaque escapes out, the artery wall may distend under physiological blood pressure to improve blood flow through the artery.

In accordance to the preferred embodiment of the present invention, the ablating apparatus is a laser ablating apparatus as shown in FIG. 4 emitting a femto-second pulsed laser having pulses preferably between 10 to 750 fs duration. The laser ablating apparatus (240) comprises a laser source (241) for emitting laser and a robotic arm (243) having reflective mirror. A nano-positioning translation stage (250) is adapted at the distal end of the robotic arm (243) and the reflecting mirror (B) is mounted on the nano-positioning translation stage (250) for directing the laser to ablate the diseased artery (100). The robotic arm (243) can travel to and fro along the axis of the artery for longitudinally ablating the diseased artery. The nano translation stage (250) is adapted to travel to and fro along the axis of the artery for longitudinally ablating the diseased artery without re-positioning the robotic arm (243). The reflective mirror B is responsible for delivery of the right amount of laser energy at the right target on the surface of the target artery. The reflective mirror B focuses a 10 micron diameter laser spot on the target of the diseased artery. Each laser pulse emits 3 to 10 joules/sq cm energy, pulse duration 10 to 750 fs and removes about 1 micron depth of tissue. Alternatively, ablation apparatus can be a mechanical ablation device including at least one blade (not shown) mounted on at least one pressure sensing transducer (not shown) for ablating the artery according to the present invention.

According to the preferred embodiment of the present invention as shown in FIG. 4, the OCT beam scanners that is longitudinal beam scanner (not shown) and the transverse beam scanner (230A, A) are adapted on the nano translation stage (250). According to the preferred embodiment of the present invention, the OCT system (230) of the present invention comprises two transverse beam scanner adapted on either side of the ablating laser beam (150) on the nano-positioning translation stage (250) for closely monitoring ablation process wherein first transverse beam scanner (230A, A) provides information related to pre-ablation and second transverse beam scanner provides post ablation information to the controller means (220) thereby allows close monitoring of the ablation process to the surgeon.

According to the present invention, the video camera (not shown) can be mounted on or near the tip of the robotic arm for monitoring the progress of ablation on the display.

The femto-second pulsed laser is precisely controlled for laser ablation of the diseased artery to expose the atherosclerotic plaque present in the arterial wall to the natural defense system of the body. The ablating laser energy is delivered to the target artery through a robotic arm guided by real time Optical Coherence Tomography (OCT) and digital image tomography, the apparatus performs high resolution depth resolved incisions over the prescribed areas of the artery.

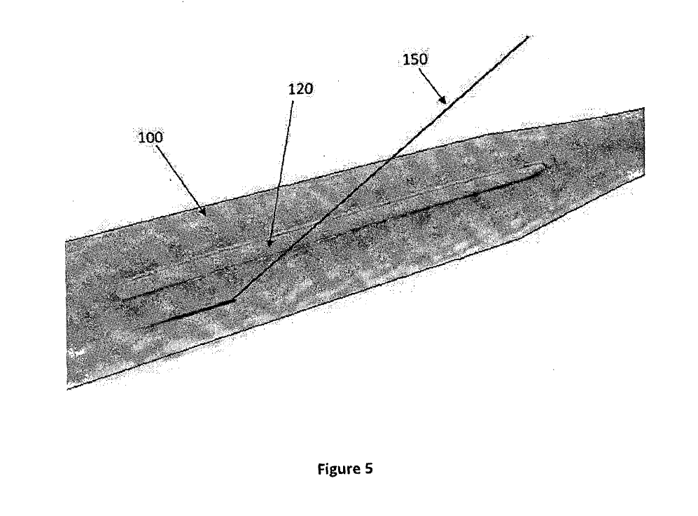

Referring FIG. 5 shows a longitudinal laser incision (120) created with the help of laser (150) by using ablating apparatus as shown in FIG. 4 to the diseased artery (100) which is along the length of the artery and may extends from one end to other end of atherosclerosis (not shown). The laser incision (120) passes through the external layer of the artery wall, the tunica adventitia (30), and tunica media (20) and through the fibrous capsule (40) exposing the plaque (50) to the body defense cells—the WBC's (macrophages, monocytes, neutrophils etc.). According to the present invention, the laser incision can be a single or multiple longitudinal incisions. Advantageously, the longitudinal incision can be a continuous or discontinuous incision and may extend from one end to another end of atherosclerosis depending upon the atherosclerosis spread in the artery or artery tree.

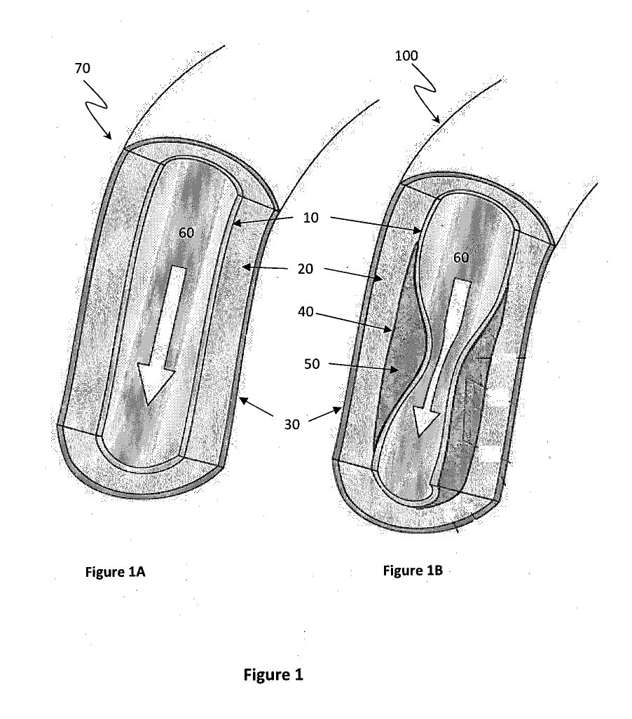

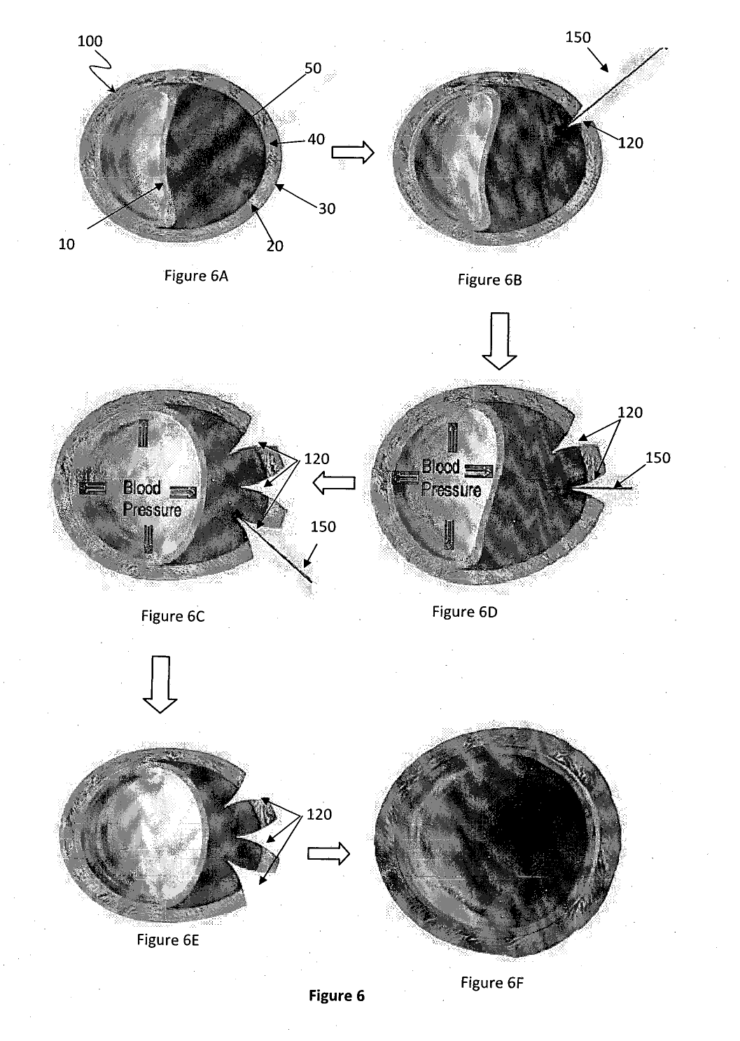

FIG. 6A shows a cross sectional view of the diseased artery (100) of FIG. 1.

FIGS. 6B-6D show cross sectional views of ablated diseased artery according to the present invention wherein multiple ablated incisions (120) created on the artery (100) one after one by laser (150) passing through tunica adventitia (30), tunica media (20) of artery and the fibrous capsule (50) in contact with the tunica media (20) and effects of laser cuts made on the surface of thickened diseased arterial wall according to the present invention due to the blood pressure inside the vessel. On ablation/incision, the contents plaques are exposed to the natural defense of the body and are destroyed by the natural defense system. The plaque escaping out of the artery on the external surface of the artery may be wiped or washed away with saline during or after the ablation procedure. Then, natural healing of artery is allowed which eliminates atherosclerosis thoroughly. FIG. 6E shows an artery healed within 4 to 6 weeks after the intervention according to the present invention.

According to the present invention, the pulse duration of laser is shorter than the thermal conductivity of tissues and preferably 10 to 750 fs. Hence, during ablation (removal of tissue) no collateral thermal injury occurs to adjacent tissues and the cells therein. This avoids post-operative scarring and re-blockage.

The apparatus of the present invention can be easily modified to integrate with endoscopic (thoracoscopic) instruments for use inside the chest cavity. The apparatus is meant to treat all the major arteries in the body that are susceptible to atherosclerosis i.e. the carotids—that supply the brain (superficial artery in the neck), the coronaries—that supply the heart and the Ilio-femorals—that supply the legs and the generative organs. The treatment method can be used for preventing stroke (of brain and heart) by treating thickened arteries before they get completely clogged or suffer thrombosis.

According to the present invention, the incision is carried out through fibrous capsule along the length of the artery/vessel to expose the plaque elements to body defense cells, white blood cells, macrophages etc. for mobilization that facilitate elimination of the contents of the plaque. As the incision of the artery wall and this fibrous capsule is from the outside of the vessel wall, the contents of the plaque escapes outside of the vessel are harmless.

This method makes possible to treat arteries of all sizes including small vessels including internal diameter less than 1 mm. Vessels with low blood flow (such as the Internal Iliac and infra-popliteal arteries) can be treated without risk of thrombosis since the artery/vessel lumen is intact and tunica intima, the arterial anti-thrombotic surface, remains undisturbed. In addition, the procedure may be carried out without diverting the blood flow through the artery. Further, the present invention treats or approaches the artery from the external side, there is no damage to the tunica intima of the artery.

Further, this invention achieves elimination of atherosclerosis in the whole regional distribution of disease thereby avoids new obstructions arising in the regional distribution of disease. Moreover it avoids re-blockage of the artery as it eliminates the atherosclerosis and also restores the pulsate character of the artery by natural way.

As the skilled person will appreciate, other types of ablating apparatus may be used according to the ablation technique employed. Such techniques include electromagnetic, photonic and ultthsound ablation.

Numerous variations may be made to the described embodiments. It is intended to include all such variations and modifications which fall within the spirit and scope of the present invention.

1-17. (canceled)

18. A method for eliminating atherosclerosis from an artery having tunica adventitia, tunica media and tunica intima wherein the atherosclerosis is formed between tunica media and tunica intima in a fibrous capsule, said method comprising step of:
approaching a diseased artery from external side; and
exposing atheromatous plaque of the atherosclerosis to the natural defense system of body inside the fibrous capsule and wiping atheromatous plaque escaping out of the artery on external surface by ablating tunica advertitia and tunica media of the artery and a fibrous capsule of the atheroscelerosis thereby eliminating atherosclerosis.

19. A method as claimed in claim 18, further comprising a step of surveying thickness of the arterial wall and length of the atherosclerosis spread in the artery for calculating length and depth of ablating incision required to expose atheromatous plaque.

20. A method as claimed in claim 19, wherein a step of surveying thickness of the arterial wall includes selection of thickest wall of the artery for ablating.

21. A method claimed in claim 18, wherein the step of ablating includes providing a single or multiple longitudinal incisions.

22. A method as claimed in claim 20, wherein the longitudinal incision can be a continuous or discontinuous incision extending from one end to another end of atherosclerosis.

23. A method as claimed in claim 18, wherein the step of ablating is carried out using a laser,

24. A method as claimed in claim 20, wherein the laser is a femto-second pulsed laser having pulses preferably between 10 to 750 fs duration.

25. An apparatus for eliminating atherosclerosis from an artery, comprising:
a surveying means having at least one longitudinal surveying scanner for providing length of the atherosclerosis and one transverse surveying scanners for providing cross section view, said surveying means surveys a thickness of an arterial wall and length of the atherosclerosis spread in the artery for defining ablating target on the surface of the artery and calculates depth of ablating incision required from external surface of the artery to a fibrous capsule of the atherosclerosis for exposing atheromatous plaque to a natural defense system;
an ablating apparatus comprising an ablating means for ablating the arterial wall externally and a robotic arm for adapting the ablating means for carrying out ablation of arterial wall;
a video camera mounted at the tip of robotic arm for monitoring the progress of ablation; and
a controller means for controlling the ablation of the arterial wall by the robotic arm, said controller means being interacting between said surveying means and ablating apparatus and being responsive to a signal from said surveying means, the signal relating to the depth and length of ablating incision required to expose atheromatous plaque to the natural defense system of the body inside the fibrous capsule helping to eliminate atherosclerosis from the artery.

26. The apparatus as claimed in claim 25, wherein said surveying means includes means to produce a 3D image of the arterial wall.

27. The apparatus as claimed in claim 26, wherein said surveying means is selected from a group of surveying means consisting of: mechanical imaging, ultrasound, magnetic resonance imaging, computed tomographic imaging using electromagnetic radiation, photonic imaging and optical coherence tomography.

28. The apparatus as claimed in claim 25, wherein the surveying means comprises two transverse surveying scanners adapted on the robotic arm before and after ablating means for monitoring ablating process in real time.

29. The apparatus as claimed in claim 25, wherein the ablating means is a femto-second pulsed laser.

30. The apparatus as claimed in claim 29, wherein the pulsed laser is of 10 to 750 fs duration and produces energy 3-10 Joules/sq cm.

31. The apparatus as claimed in claim 29, wherein the pulsed laser has a diameter of about 10 micron.

32. The apparatus as claimed in claim 25, wherein the ablating means includes a mechanical ablation device including at least one blade mounted on at least one pressure sensing transducer.

33. A method as claimed in claim 18, wherein the step wiping atheromatous plaque escaping out of the artery includes washing atheromatous plaque escaping out of the artery with saline.
